(19) United States
(12) Patent Application Publication
(10) Pub. No.: US 2026/0229350 A1
(43) Pub. Date: Aug. 6, 2026

(54) SEIZURE PREDICTION SYSTEM THAT COMBINES BRAIN ELECTRICAL DATA AND CARDIORESPIRATORY DATA

(71) Applicant: NIHON KOHDEN DIGITAL HEALTH SOLUTIONS, LLC, Irvine, CA (US)

(72) Inventors: Joshua Andrew EHRENBERG, San Diego, CA (US); Harsh DHARWAD, San Diego, CA (US); Abel LIN, San Diego, CA (US); Timothy RUCHTI, San Diego, CA (US)

(73) Assignee: NIHON KOHDEN DIGITAL HEALTH SOLUTIONS, LLC, Irvine, CA (US)

(21) Appl. No.: 19/044,830

(22) Filed: Feb. 4, 2025

(51) Int. Cl. G16H 50/20; A61B 5/00; A61B 5/0205; A61B 5/11; A61B 5/353; A61B 5/355; A61B 5/358; A61B 5/369; G16H 10/60

(52) U.S. Cl. CPC G16H 50/20; A61B 5/0205; A61B 5/11; A61B 5/353; A61B 5/355; A61B 5/358; A61B 5/369; A61B 5/4094; G16H 10/60

(57) ABSTRACT

A system that calculates the probability that a patient will experience a seizure using a machine learning system. Inputs to the machine learning system may include ECG, EEG, vital signs, lab results, and patient history and demographics. Features may be extracted from the raw data and input into the machine learning system. Neurological features calculated from EEG waveforms may include characterizations of discharges, such as their frequency, duration, amplitude, and shape. Cardiovascular features calculated from ECG waveforms may include characterizations of P, Q, R, S, T waveforms, and heart rate variability metrics. Relationships between neurological and cardiovascular features may also be calculated and input into the system; these relationships may include for example correlations, cross-coherence, wavelet correlation, and cross-spectral entropy. The seizure risk probability may be updated periodically as new patient data arrives. Combining multiple data sources across body subsystems may increase the accuracy of seizure prediction.

### BACKGROUND OF THE INVENTION

### Field of the Invention

One or more embodiments of the invention are related to the field of health care information systems and medical devices. More particularly, but not by way of limitation, one or more embodiments of the invention enable a seizure prediction system that combines brain electrical data and cardiorespiratory data.

### Description of the Related Art

Clinicians apply heuristics to detect or classify many specific types of patient conditions. These heuristics are often applied to inform non-specialists when to apply a specific treatment. Detailed analysis of subtle changes typically requires specialists, who may review data too late to intervene.

Existing heuristics are often inadequate for several reasons: (1) They are by necessity highly simplified to support rapid, manual decision-making by generalists, and therefore they may ignore subtle changes and signs. (2) They generally focus on data from a single body subsystem, such as the cardiovascular system, and may therefore not incorporate data from multiple subsystems simultaneously. (3) They often focus on detecting serious conditions, rather than on predicting them; interventions that may prevent patient deterioration may therefore not be made in time. Once a critical event occurs, it may be difficult or impossible to treat it.

A specific example of a critical condition that could be potentially predicted is seizure. There are no known systems that continuously monitor patient data from multiple subsystems (in particular, brain and cardiorespiratory) to predict the occurrence of a seizure. With advance warning of an impending seizure, clinicians could intervene early and potentially prevent it or mitigate its effects.

For at least the limitations described above there is a need for a seizure prediction system that combines brain electrical data and cardiorespiratory data.

### BRIEF SUMMARY OF THE INVENTION

One or more embodiments of the invention may enable a seizure prediction system that combines brain electrical data and cardiorespiratory data. The system may employ a machine learning system to learn the seizure risk from a training dataset that includes patients who did and who did not experience seizures.

One or more embodiments of the invention may include one or more processors that are coupled to: an electroencephalogram monitor coupled to a patient that measures continuous electroencephalogram waveforms over time, an electrocardiogram monitor coupled to the patient that measures continuous electrocardiogram waveforms over time, and one or more vital sign sensors coupled to the patient that measure one or more vital signs over time. It may include a machine learning system that executes on at least one of the processors. The machine learning system may be trained to calculate the probability that the patient will experience a seizure within a prediction time period. The system may include a feature extraction system that executes on at least one of the processors. The feature extraction system may calculate one or more features that include: one or more neurological features calculated from the electroencephalogram waveforms over time, one or more cardiovascular features calculated from the electrocardiogram waveforms over time, one or more neurological-cardiovascular relationships calculated from one or more feature pairs—each pair with a neurological feature and a cardiovascular feature, and one or more vital sign features calculated from the one or more vital signs. The feature extraction system may input the features into the machine learning system.

In one or more embodiments, the machine learning system may be further trained to calculate the probability that said patient will experience, within the prediction time period, one or more of: sepsis, cardiogenic shock, hypotension, respiratory failure, hemorrhage, stroke, fatal arrythmia, vasospasm, and cardiac arrest.

In one or more embodiments, calculation of neurological features may include identifying one or more discharges within the electroencephalogram waveforms, and calculation of: prevalence of the one or more discharges, duration of the one or more discharges, frequency of the one or more discharges, phases of the one or more discharges; sharpness of the one or more discharges; amplitude of the one or more discharges; polarity of the one or more discharges, and the inter-discharge period between the one or more discharges. In one or more embodiments the neurological features may also include periodicity of the one or more discharges, and morphology of the one or more discharges.

In one or more embodiments, the one or more processors may be further coupled to one or more cameras configured to capture video of patient movement; and the one or more neurological features may be further calculated from this video of patient movement.

In one or more embodiments, calculation of the cardiovascular features may include identifying segments within one or more beats of the electrocardiogram waveforms over time, where the segments include a P-wave, a Q-wave, an R-wave, an S-wave, and a T-wave. It may include calculation of the amplitude of the segments, calculation of the intervals of the segments, calculation of the ST elevation from the S-wave and the T-wave, and calculation of one or both of the power spectral density and principal components scores from one or both of the amplitudes of the segments and the intervals of the segments. In one or more embodiments, the cardiovascular features may also include arrythmia frequency and one or more heart rate variability metrics. The heart rate variability metrics may include the percentage of power in the high frequency range, sample entropy, the local dynamics score, and the coefficient of sample entropy.

In one or more embodiments, the one or more neurological-cardiovascular relationships may include one or more of: a correlation between a neurological feature and a cardiovascular feature, a cross-coherence between a neurological feature and a cardiovascular feature, a wavelet correlation between a neurological feature and a cardiovascular feature, and a cross-spectral entropy between a neurological feature and a cardiovascular feature.

In one or more embodiments, the vital signs may include respiratory rate, heart rate, blood oxygenation, blood pressure, and temperature. In one or more embodiments the vital sign features may include the mean, median, standard deviation, and trend of each vital sign; the correlation coefficient between heart rate and respiratory rate; and the correlation coefficient between heart rate and blood oxygenation. In one or more embodiments the vital sign features may also include one or more blood pressure variability metrics, which may include multiscale sample entropy of the blood pressure.

In one or more embodiments, the features may also include one or more lab results associated with the patient, one or more values of patient history and demographics associated with the patient. The lab results may include results from a metabolic panel, results from a complete blood count, and the neuron-specific enolase level. Patient history and demographic values may include age, sex, history of seizures, history of epilepsy, history of head trauma, and history of neurological infection.

In one or more embodiments, the machine learning system may include one or more of: an XGBoost model, a support vector machine model, a logistic regression model, a random forest model, and a neural network model.

### BRIEF DESCRIPTION OF THE DRAWINGS

The above and other aspects, features and advantages of the invention will be more apparent from the following more particular description thereof, presented in conjunction with the following drawings wherein:

FIG. 1 illustrates a typical process used in the prior art to manage patients at risk for one or more critical events: multiple clinicians may receive patient information from a number of different sources and may attempt to detect the onset of critical events within their area of expertise or responsibility.

FIG. 2 shows an overview architectural diagram of an illustrative embodiment of the invention, which uses a machine learning system to integrate patient data from multiple devices and body subsystems in order to predict the likelihood of a critical event.

FIG. 3A shows a variation of the system of FIG. 2, in which the inputs to the machine learning system are waveform or other data received directly from devices.

FIG. 3B shows a different variation of the system of FIG. 2, in which the inputs to the machine learning system are engineered features extracted from device data.

FIG. 4A illustrates an embodiment of the invention that updates the probability of an event over time as device data is received.

FIG. 4B shows an illustrative display of the change in risk over time for a specific patient, which shows risk for seizure, sepsis, and mortality.

FIG. 5 shows illustrative engineered statistical features that may be used in one or more embodiments of the invention; features may be based on a single time series of data, or on comparisons between different time series.

FIG. 6 shows an illustrative embodiment of the invention that predicts the risk of a seizure using features obtained from EEG waveforms, vital signs, ECG waveforms, lab results, and patient history and demographics.

FIG. 7 shows illustrative features input into the machine learning system of FIG. 6 that may be calculated from EEG waveforms.

FIG. 8 shows some of the features of FIG. 7 for an illustrative slice of an EEG waveform.

FIG. 9 shows illustrative features input into the machine learning system of FIG. 6 that may be calculated from ECG waveforms.

FIG. 10 shows illustrative features input into the machine learning system of FIG. 6 that may be calculated from blood pressure variations.

FIG. 11 shows illustrative features input into the machine learning system of FIG. 6 that may be obtained from labs or patient history and demographics.

FIG. 12 shows illustrative machine learning techniques that may be used with the system of FIG. 6.

### DETAILED DESCRIPTION OF THE INVENTION

A seizure prediction system that combines brain electrical data and cardiorespiratory data will now be described. In the following exemplary description, numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

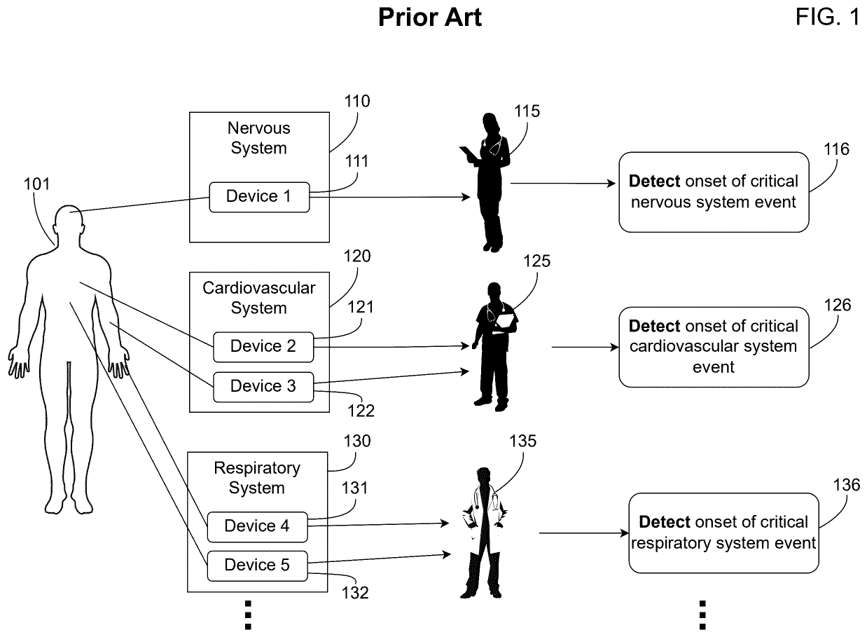

For patients at risk of developing critical complications—such as seizures, cardiac arrest, sepsis, hypotension, or cardiovascular shock—applying appropriate treatments is both complex and time critical. Current practices are generally inadequate to provide advance warning of impending critical events. This situation is illustrated in FIG. 1. Patient 101, who may be in a hospital for example, may be monitored by multiple devices across multiple body subsystems; for example, device 111 may monitor the nervous system 110, devices 121 and 122 may monitor the cardiovascular system 120, and devices 131 and 132 may monitor the respiratory system 130. These systems and devices are illustrative. Data from the devices may be provided to clinicians who are monitoring or treating patient 101. Often different body subsystems will be analyzed by different specialists; for example, data from device 111 may be reviewed by clinician 115, data from devices 121 and 122 may be reviewed by clinician 125, and data from devices 131 and 132 may be reviewed by clinician 135. Each clinician may use clinical judgement and experience to interpret the data in his or her area of expertise. Typically, clinicians are trained to recognize signs of critical events that are already occurring; for example, clinician 115 may analyze data from device 111 to make a judgement 116 that a critical nervous system event is occurring; similarly clinicians 125 and 135 may make judgements 126 and 136, respectively, in their areas of expertise.

The situation shown in FIG. 1 illustrates several shortcomings of current processes: (1) data is not integrated across body subsystems; (2) clinicians often interpret data manually based on their training and expertise, which may vary widely; (3) clinicians are generally better able to detect events than to predict them, which may delay treatment or intervention until it is too late.

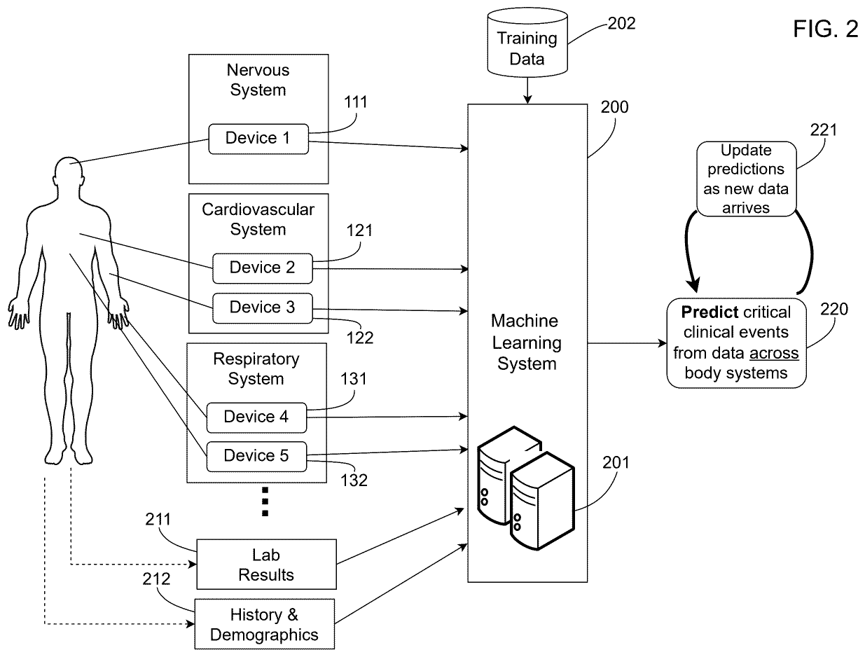

FIG. 2 shows an illustrative embodiment of the invention that addresses these shortcomings. This embodiment uses a machine learning system to integrate and interpret data across body subsystems, and it predicts one or more critical events rather than simply detecting them. Machine learning system 200, which may execute on any subset of one or more processors 201, receives data from any number of devices that measure patient data, such as devices 111, 121, 122, 131, and 132 of FIG. 1. Data may be obtained from any type or types of devices, including devices attached to the patient or that analyze information or samples received from the patient. Devices may be associated with any body subsystems, including but not limited to the nervous system, cardiovascular system, and respiratory system as shown in FIG. 2. Any number of devices may be associated with each subsystem. Some devices may measure multiple physical variables (such as a multi-lead EEG), while some may measure only a single value (such as a blood pressure monitor). Processor(s) 201 may be any type or types of computer, circuit, CPU, GPU, or a network of any of these devices.

In one or more embodiments of the invention, machine learning system 200 may also receive input from one or both of lab results 211 and patient history and demographics 212. This information may be received from a laboratory system or from an electronic medical record, for example.

Machine learning system 200 may output a prediction 220 as to whether a specific critical event will occur for the patient. This prediction may be expressed for example as a probability that the event will occur within a specified timeframe (such as within the next 2 hours). It may also be expressed as a risk category, or a relative risk (compared to a patient population, for example.) Prediction may be made for any type of event; in one or more embodiments the system 200 may output predictions for multiple types of events, or for multiple timeframes. (For example, it may output a probability of a seizure and the probability of sepsis within the next hour, next 2 hours, and next 8 hours.) The prediction (probability or other measures) may be based on the total data received from all body subsystems (and from labs and history/demographics if included). The system may therefore combine data across body subsystems, and may use relationships between subsystems, to calculate its predictions. Combining data from multiple organ systems may lead to superior prediction of various types of events. As data from devices or other sources changes, the prediction may be updated in cycle 221; for example, if the data indicates that a patient's condition is deteriorating, the prediction probability of one or more critical events may increase. Predictions 220 and updates 221 may be displayed to clinicians; in one or more embodiments the system may also provide suggested treatments to prevent development of a condition that is predicted to occur with relatively high probability.

The machine learning system 200 may be trained using a training dataset 202, which may for example contain recorded patient data labeled with whether and when the event or events of concern occurred in that patient. The training dataset 202 may be updated over time as the system is in operation, and the machine learning system may be retrained as the dataset grows.

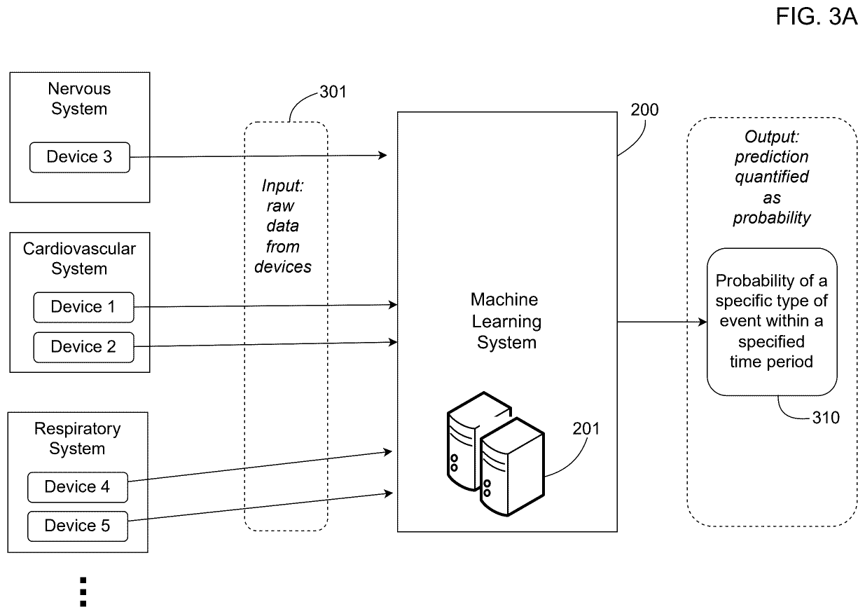

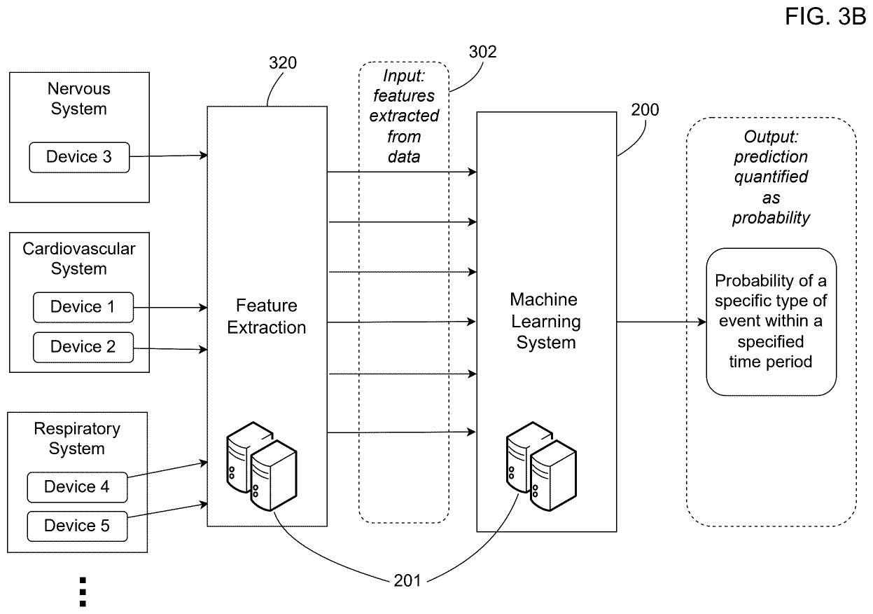

FIGS. 3A and 3B illustrate two variations of the architecture of FIG. 2. In the architecture shown in FIG. 3A, machine learning system 200 operates on inputs 301 that are obtained directly from the devices monitoring the patient, such as EEG (electroencephalogram) or ECG (electrocardiogram) waveforms for example. The system 200 outputs a probability 310 that a specific event of interest will occur in the patient within a specified time period. In the architecture shown in FIG. 3B, a feature extraction process 320 is performed on the data from the devices to generate a set of feature values 302, and these feature values are input into machine learning system 200. Combinations of the approaches of FIG. 3A and FIG. 3B may be used in one or more embodiments, where machine learning system 200 receives as inputs a combination of raw data from devices and extracted features. Extracted features may also include any of the raw data or any subsets thereof. Feature extraction 320 may execute on any subset of processor(s) 201, which may be the same as or distinct from the subset of processor(s) used by machine learning system 200.

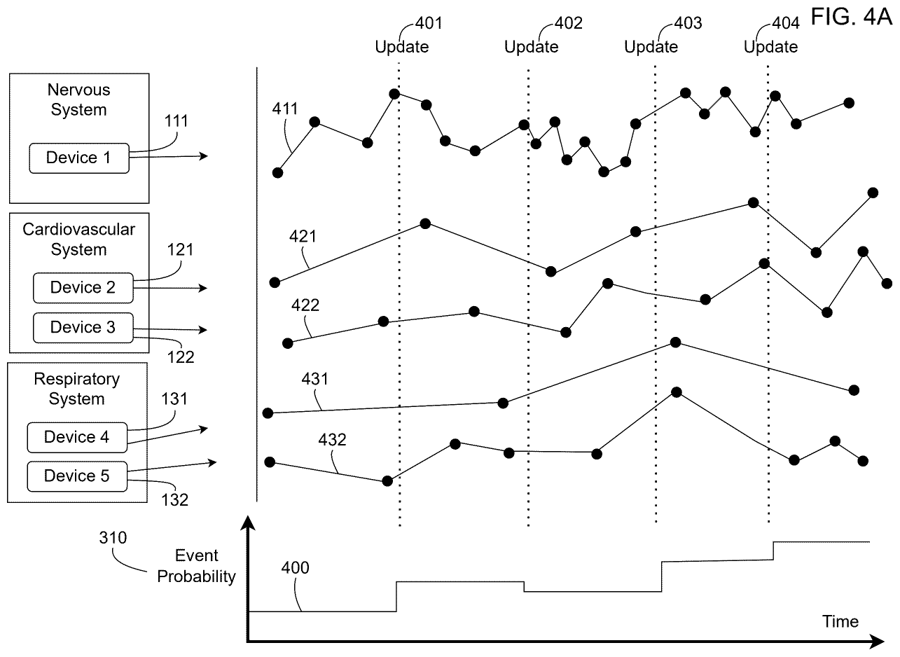

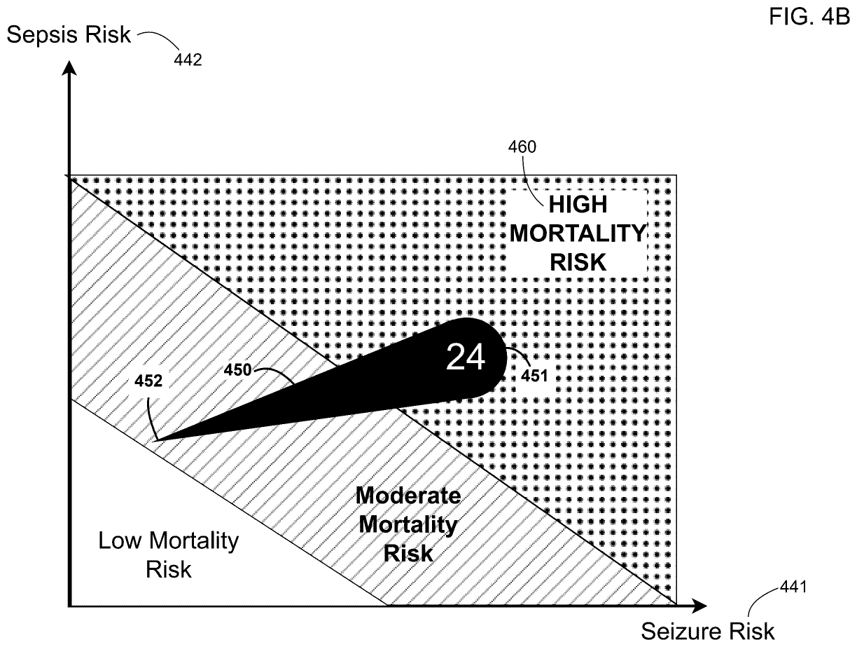

FIG. 4A illustrates updates to an event probability over time as new data is received from devices or other sources. In this simplified example, each of the devices 111, 121, 122, 131, 132 generates a corresponding time series of data values 411, 421, 422, 431, 432, respectively. (In general, a device may generate one or more time series of data.) Different time series may be updated with different frequencies; for example, values of time series 411 are updated more frequently than those of time series 431. Machine learning system 200 may calculate updated event probabilities 310 at specified time intervals: at times 401, 402, 403, 404, etc. the system calculates a new probability of the critical event (or events). The probability 400 therefore changes over time as new data becomes available. Updates such as 401 may occur at regular intervals (such as once every 5 minutes, for example), or when a specified amount of new data becomes available. Each update may be based on the data received since the previous update, and on any previous history.

In one or more embodiments, the system may include one or more graphical displays that show the evolution of patient risk for one or more serious events over time. FIG. 4B shows an illustrative plot for a specific patient, identified as patient number 24. (In some embodiments, risk trajectories for multiple patients may be shown on the same display, so it may be important to identify each patient's trajectory on the graph.) The black area 450 plots the risk trajectory for the patient on two axes: the horizontal axis 441 represents the predicted risk for seizure, and the vertical axis 442 represents the predicted risk for sepsis. Plots may be generated for any type or types of risk, including but not limited to seizure and sepsis risk. Trajectory 450 shows the change in risk (for seizure 441 and sepsis 442) over time, with the tail (narrow end) 452 representing a past time (such as 2 hours ago, for example), and the head (wide end) 451 representing the current risk. Such a plot allows clinicians to understand at a glance how the patient is evolving and what the current level of risk is for the patient. Another variable dependent on the factors 441 and 442, such as total mortality risk, may also be shown as contours or regions on the plot; for example, the current risk 451 for the patient puts the patient in the high mortality risk zone 460.

A display of risk trajectory for one or more events, as shown in FIG. 4B, is generally more valuable to clinicians than existing alarm systems that provide a binary yes/no signal to indicate when a patient has entered a critical state. Alarm systems often generate many false positives, which leads to alarm fatigue among clinicians and the potential that patients with real critical events will not be treated adequately or in time. With a risk trajectory, clinicians can continually reassess the need for interventions without the burden of responding to constant alarms.

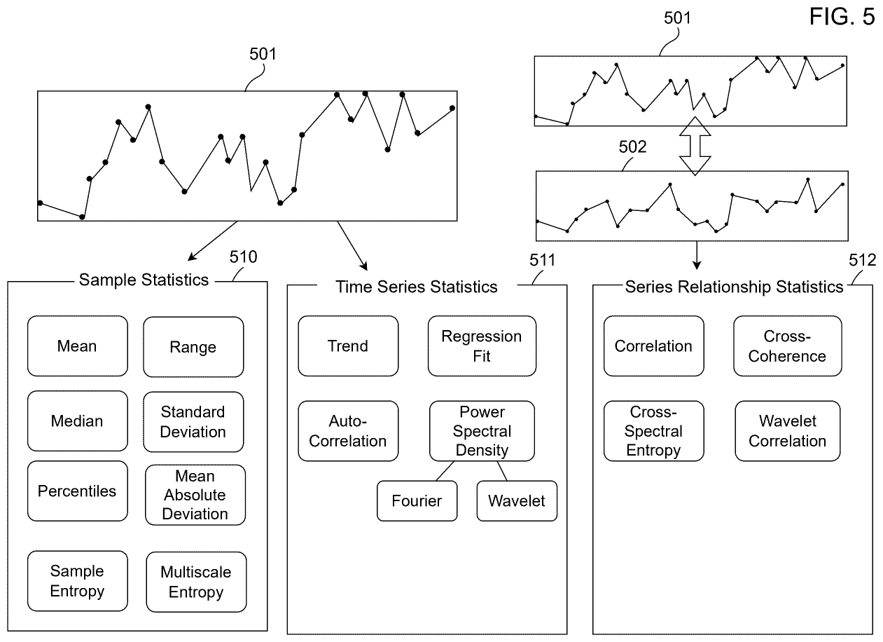

As illustrated in FIG. 3B, in one or more embodiments the data from devices or other data sources may be processed in feature extraction step 320 to calculate one or more features from the raw data, and these features may be input into the machine learning system. FIG. 5 shows some illustrative statistical features that may be calculated from any time series of data, and from combinations of any pair of time series. Any of these features may be calculated from any time series from any device or other data source. Statistics may be applied to any time slice of a time series; for example, a mean value may be calculated for a series of values over any 30-minute time slice, and this feature may be updated every 30 minutes (or as a rolling average more frequently). Time slices of different durations may be applied to different data series. In one or more embodiments, any time series may be downsampled, upsampled, filtered, smoothed, transformed, integrated, or differenced, and then statistics may be applied to the result.

FIG. 5 shows illustrative sample statistics 510 and time series statistics 511 that may be applied to a single time series 501, and series relationship statistics 512 that may be applied to any pair of time series 501 and 502. Sample statistics 510 may include for example, without limitation, the mean, median, range, standard deviation (or variance), any percentiles (such as quartiles, or 10% and 90% percentiles), mean absolute deviation, sample entropy, and multiscale entropy at any set of scales. Time series statistics 511 may include trend (for example from a linear or nonlinear regression), autocorrelation at various lags, fit statistics (such as R-squared or F) from regression, and power spectral density measurements at different frequencies resulting from Fourier or Wavelet decompositions, for example.

Statistics 512 may be applied to any pair of time series 501 and 502. Any of these statistics may be applied to any comparable time slices of these series, or to entire time series containing all received values. These statistics may measure the degree or type of relationships between the two series. Illustrative relationship statistics may include for example, without limitation, correlation, cross-coherence, wavelet correlation, and cross-spectral entropy. Correlation may be measured via a correlation coefficient, cross correlation, or cross-variance. Cross-coherence quantifies the degree of linear correlation or synchronization between two signals as a function of frequency and is determined using the cross-spectral density and the power spectral density of two signals. Given two signals, x(t) and y(t), Fourier transforms of the signals yield X(f) and Y(f) , respectively. The power spectral density is determined by multiplying X(f) and Y(f) by their respective conjugate: Sₓₓ(f)=X(f)X*(f) and S_{yy}(f)=Y(f)Y*(f) The cross spectral density, S_{xy}(f) , is defined as S_{xy}(f)=X(f)Y*(f) , and the cross-coherence is given by C_{xy}(f)=|S_{xy}(f)|²/(Sₓₓ(f)S_{yy} (f)). Wavelet cross-correlation is the cross-correlation between wavelet coefficients of two signals, calculated via a continuous wavelet transform of two signals through time as a function of scale. Cross-spectral entropy quantifies the complexity or randomness of the relationship between two signals in the frequency domain; it is defined by

$${H}_{xy} ( f ) = - {\sum }_{f} {P}_{xy} ( f ) \cdot log {P}_{xy} ( f ) ,$$

where P_{xy}(f) is the normalized cross-spectral density.

In one or more embodiments, relationships may be measured between data series corresponding to different body subsystems. Such relationships may have substantial predictive value, because they provide insight into the body's overall physiological function. The body's dynamic physiological systems operate together in a seamless manner to supply function, performance and health. When disease occurs, compensatory mechanisms are activated to protect vital organs, and particularly the brain, from damage such as ischemia. In this circumstance, the variability within and between all systems is reduced as their action becomes entrained. Consequently, evaluation, detection and prediction of health-related events is significantly improved by combining sensor measurement data representing all significant physiological systems.

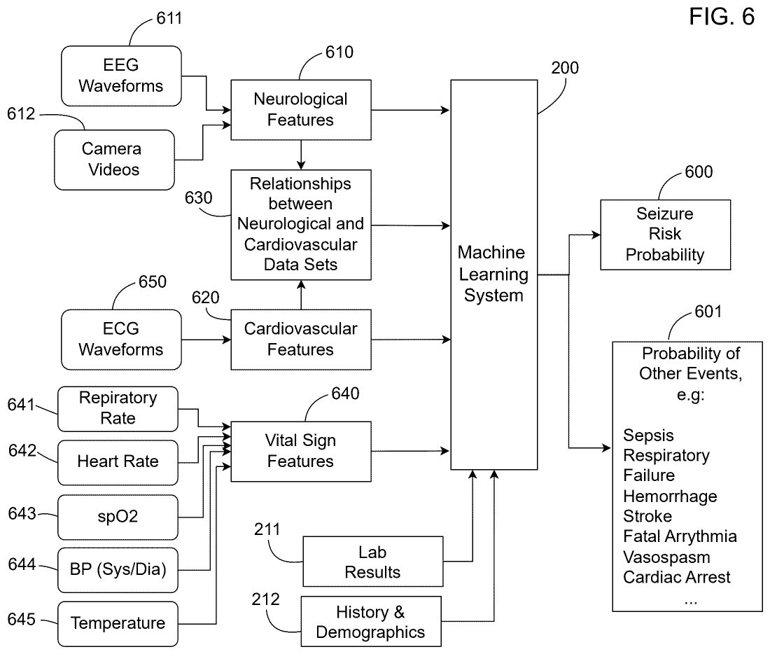

The machine learning systems described above may be applied to calculate the risk of a patient developing any critical condition or experiencing any critical event of interest. FIG. 6 shows an illustrative machine learning system 200 that may be applied for example to predict the risk 600 of a seizure. This risk 600 may be expressed for example as the probability that the patient being monitored will experience a seizure within some fixed time period, such as within the next two hours. In this illustrative embodiment, the inputs to machine learning system include neurological features 610 calculated from EEG waveforms 611 received from an EEG monitor (which may be incorporated into a cardiorespiratory bedside monitor) coupled to the patient, cardiovascular features 620 calculated from ECG waveforms 650 received from an ECG monitor coupled to the patient, and vital sign features 640 calculated from vital signs received from vital sign sensors coupled to the patient (such as bedside monitors). Vital signs used by the machine learning system may include for example, without limitation, respiratory rate 641, heart rate 642, spO2 (blood oxygen) 643, blood pressure 644 (systolic and diastolic), and temperature 645. A potential application of such a system is for monitoring and treatment of a patient that is hospitalized with bedside monitoring that includes measurement of these inputs over time. As described above with respect to FIG. 5, relationships between different body subsystems may also be valuable inputs; the system of FIG. 6 therefore includes inputs 630 that are pairwise statistical relationships (such as statistics 512 of FIG. 5) between any or all of the cardiovascular features (which may include any of the raw data received from cardiovascular sensors) and any or all of the neurological features (which may include any of the raw data received from cardiovascular sensors). Relationships between any of the vital sign features 640 and any of the neurological or cardiovascular features may also be inputs into the machine learning system. Combining cardio-pulmonary (bedside) monitor data and neurological measurements provides a significant opportunity to improve the sensitivity of detection algorithms and the predictive capability of machine learning models.

In one or more embodiments of the invention, neurological features 610 may also be extracted from video 612 captured by one or more cameras that observe the patient. These video features may be combined with EEG data and other physiologic variables to further enhance seizure prediction and detection accuracy and to assist in diagnosis of seizure type. In epileptic seizures there is often a characteristic stereotyped physical movement. This is why video is routinely recorded with EEG in EMUs. In one or more embodiments a LIDAR camera may be used, and video data 612 may include point clouds or other three-dimensional data that may facilitate analysis of patient movements; however, even in a normal 2D video, many stereotyped and rhythmic movements may be detectable.

A sequence of images, acquired via video cameras or LIDAR-based scanning systems, may be collected to capture spatial and temporal data describing a patients'posture and motion. From these image sequences, specialized morphological and kinematic features may be extracted, including the precise location (i.e., two-or three-dimensional coordinates) of key anatomical landmarks such as the head, shoulders, arms, and legs. In addition, facial features may be derived using detection algorithms, such as Haar Cascades or convolutional neural networks trained on large-scale facial datasets, to localize the face and identify individual facial components (e.g., eyes, nose, chin, mouth). Once the salient features have been detected and characterized, their temporal evolution may be quantified by calculating position trajectories and deriving instantaneous velocities and accelerations. These time series of features, associated with anatomical and morphological locations and position, velocity and acceleration, may then be normalized by removal of the dominant trend to ensure consistency between patients and to remove artifacts associated with movement, and analyzed using time series algorithms aimed at further characterizing the nature of the movement. Features describing the movements may include sample entropy, the coefficient of sample entropy, multi-scale entropy, approximate entropy, and wavelet-based multi-resolution decompositions. These methods characterize the complexity, regularity, and spectral properties of the extracted feature signals. The resulting features and derived entropy-based complexity measures may be input into machine learning system 200 along with features from EEG and cardiorespiratory monitoring data, to predict the likelihood of neurological events, including seizure. Combining all these data sources-movement features derived from video, neurological features derived from EEG, and cardiorespiratory features-may improve the accuracy and robustness of seizure prediction algorithms

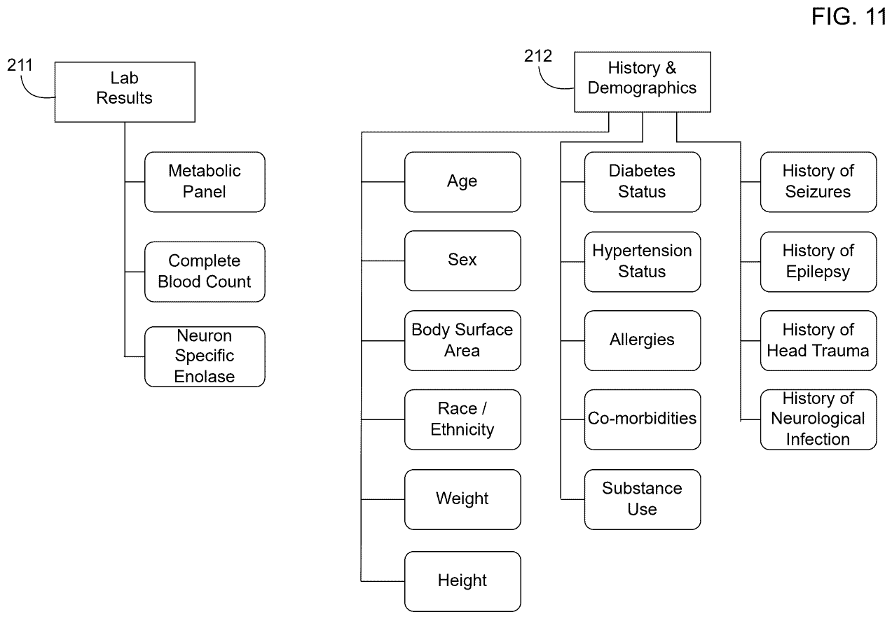

In one or more embodiments, the system 200 of FIG. 6 may also receive lab results 211 and patient history and demographics 212 as inputs; illustrative lab and history/demographic inputs are described below with respect to FIG. 11.

A system such as machine learning system 200 that combines neurological and cardiorespiratory data may also be used to predict the probability of various other events 601 besides or in addition to seizure. These events may include for example, without limitation, sepsis, cardiogenic shock, hypotension, respiratory failure, hemorrhage, stroke, fatal arrythmia, vasospasm, and cardiac arrest. For each of these events, signatures of impending problems may be visible in both neurological and cardiorespiratory data, and in the relationships between these subsystems. For example, measurements from the cardiorespiratory system may include subacute prodromes that precede seizure. Consequently, the combination of EEG and cardiorespiratory monitoring data that is illustrated in FIG. 6 provides significant advantage to seizure prediction. However, EEG data contains subacute prodromes that reflect the deterioration of the cardiovascular system. Consequently, the same methodology provides significant advantage in predicting sepsis (or other critical events).

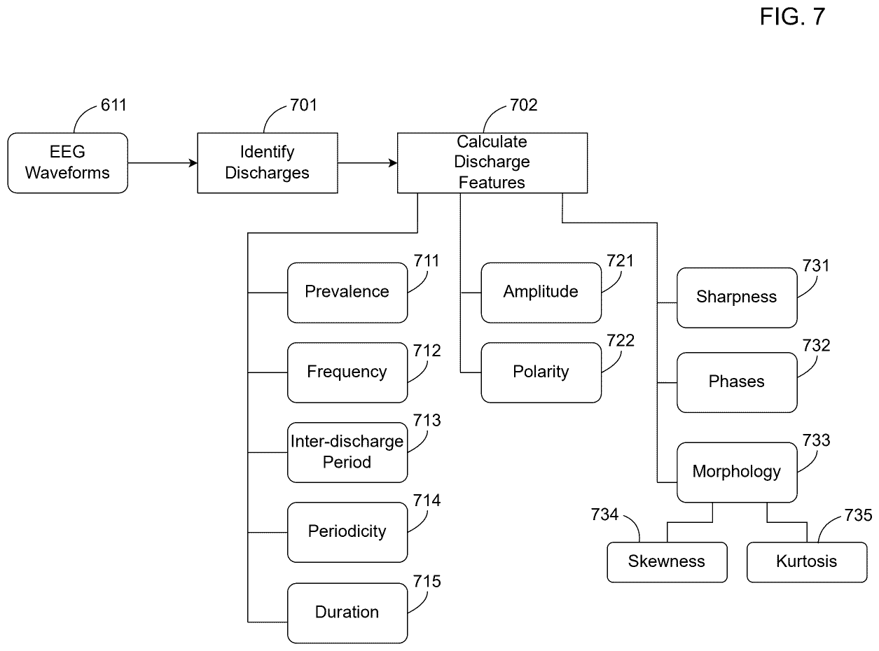

FIG. 7 shows illustrative neurological features that may be extracted from EEG waveforms 611 and may be input into machine learning system 200. In one or more embodiments, the waveforms themselves may also be input directly into the machine learning system. Any of the statistical measures shown in FIG. 5 may also be applied to any of the features shown in FIG. 7 (and to the EEG waveforms directly), and any of the resulting statistics may also be input into the machine learning system 200. In the feature extraction process shown in FIG. 7, EEG waveforms 611 are first analyzed in step 701 to identify discharges. A discharge is a sudden and brief surge of electrical activity in the brain, observed as a spike or wave on an EEG waveform, indicative of neuronal firing and often associated with epileptiform activity or other neurological abnormalities. The discharges are of various types that are distinguished by their characteristics and clinical significance. For example, periodic lateralized epileptiform discharges are sharp waveforms or spikes that appear periodically (every 1-2 seconds), usually over one hemisphere of the brain. These are often seen in patients with acute brain injuries like strokes, tumors, or infections. Generalized periodic discharges (GPDs) are characterized by periodic sharp waveforms or spikes that are seen across both hemispheres, usually occurring at regular intervals. The properties and characteristics of discharges through time provide a means to quantify the risk associated with potential neurological events.

Once discharges are identified, step 702 may calculate neurological features 610 from the EEG waveforms with their discharges. These features may quantify the risk associated with potential neurological events. Calculated features may include for example, without limitation: Prevalence 711: the proportion or percentage of time within the pre-seizure period that periodic discharges are present. Frequency 712: the number of discharges occurring per unit of time. Inter-discharge period 713: the time period between discharge events. Periodicity 714: the regularity of discharge patterns through time determined based upon the consistency of interval timing. Duration 715: the length of time that each discharge lasts. Amplitude 721: the measure of the height or magnitude of the discharge wave, measured for example in microvolts (μV). Polarity 722: the direction of the waveform deflection, where positive polarity means the waveform peaks above the baseline, while negative polarity means it peaks below the baseline. Sharpness 731: the rate of change in the waveform determined from the slope. Phases 732: the segments within each discharge cycle including components such as the onset, peak, and offset of the wave. Morphology 733: metrics derived from the shape of individual discharges, including for example Skewness 734 and Kurtosis 735.

The features are computed through time producing a time series and compared to other time synchronous activity and relative to baseline activity. The time series of metrics may be further analyzed by calculating any statistical values from the time series, such as the average, range, trend, variability (standard deviation and standard deviation of first differences), power spectral density determined for example from a periodogram, sample entropy, and multi-scale entropy.

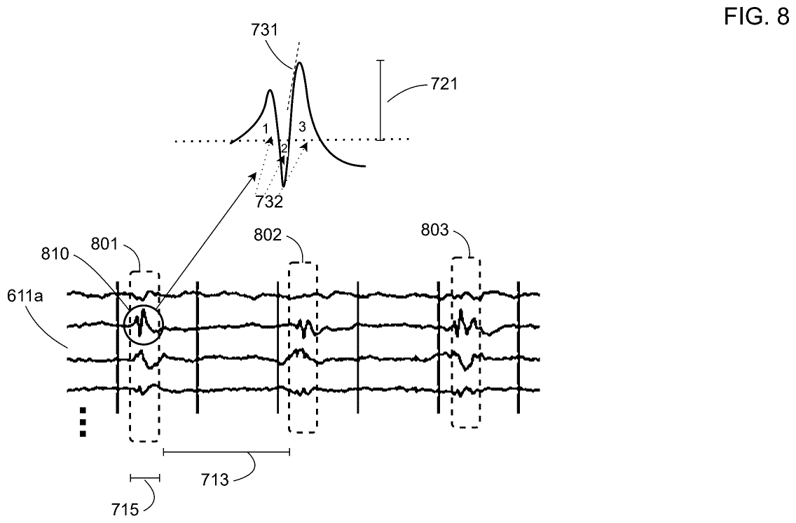

FIG. 8 shows EEG waveforms 611a that illustrate some of the features described with respect to FIG. 7. Discharges are identified at time periods 801, 802, and 803; these discharges clearly show high periodicity 714. Discharge 801 has duration 715 and inter-discharge period 713 to the next discharge 803. The reciprocal of the sum of time periods 715 and 713 is the discharge frequency 712. Illustrative waveform 810 during discharge 801 shows three distinct phases 732. It has sharpness (slope) 731, amplitude 721, and the polarity 722 is mixed but primarily positive.

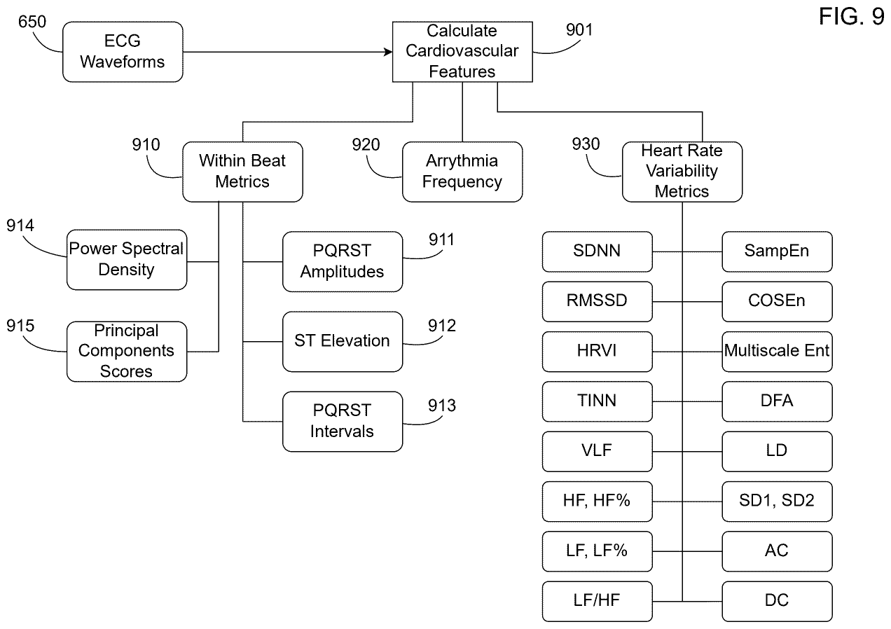

FIG. 9 shows illustrative cardiovascular features that may be calculated from ECG waveforms 650 and may be input into machine learning system 200. In one or more embodiments, the waveforms themselves may also be input directly into the machine learning system. Any of the statistical measures shown in FIG. 5 may also be applied to any of the features shown in FIG. 9 (and to the ECG waveforms directly), and any of the resulting statistics may also be input into the machine learning system 200. Step 901 may calculate features from the ECG waveforms 650; illustrative features may include within-beat metrics 910, frequency 920 of arrythmia events (such as premature ventricular contractions), and various hear rate variability metrics 930.

Within-beat features 910 may analyze a sequence of heartbeats by detecting the standard P, Q, R, S and T waveforms within each beat. Features 911 may measure the amplitudes of each PQRST point from baseline. Features 913 may measure the lengths of each of these PQRST intervals. Feature 912 measures the amplitude from baseline to the point where the ST segment of an electrocardiogram deviates upward.

Power spectral density 914 may be calculated for any or all of the ECG intervals determined through either a continuous or discrete wavelet decompensation (e.g. either using a Haar, Daubechies or Symlet Wavelet) or a Fourier transform over the entire window. Scores 915 may be calculated from a principal component decompensation of the time segments associated with each of the beats within the window, for example after normalizing the amplitude to the peak value and subtracting the baseline average (of each).

Heart rate variability features 930 may be derived from ECG R-R intervals and normalized R-R intervals. Analyses may be conducted, for example, over two-minute time windows. When the focus is variability, the length of R-R intervals may be standardized by dividing each interval by the average R-R interval within the time window. Time-domain and frequency-domain statistics may be applied to the resulting intervals. Given the non-uniform sampling of the set of R-R intervals, the frequency domain metrics may be calculated for example on the basis of the Lomb-Scargle Periodogram.

Illustrative heart rate variability features 930 may include, for example, without limitation: SDNN: the standard deviation of NN intervals. RMSSD: the root mean square of successive differences. HRVI: the HRV triangular index (calculated for example as the total number of all NN intervals divided by the height of the histogram of all NN intervals measured on a discrete scale with bins of 7.8125 ms (1/128 seconds)). TINN: RR histogram baseline width (baseline width of the minimum square difference triangular interpolation of the highest peak of the histogram of all NN intervals). VLF: absolute very low frequency power to the range of 0.003 Hz to 0.04 Hz. LF: low frequency power in the range of 0.04 Hz to 0.15 Hz. HF: high frequency power in the range of 0.15 Hz to 0.4 Hz. LF %: low frequency percent power. HF %: high frequency percent power. LF/HF: the ratio between low and high frequency power. SampEn: the sample entropy. Multiscale sample entropy: including the slope of Sample Entropy vs Scale for scales 1-5, the average of scales 1-5, and the ratio of the predefined slope and average. DFA: detrended fluctuation analysis. LD: local dynamics score. COSEn: coefficient of sample entropy (sample entropy normalized to heart rate). SD1, SD2 and their ratio derived from a Poincare plot which plots each R-R interval against the next. AC: the heart rate acceleration capacity index. DC: the heart rate deceleration index.

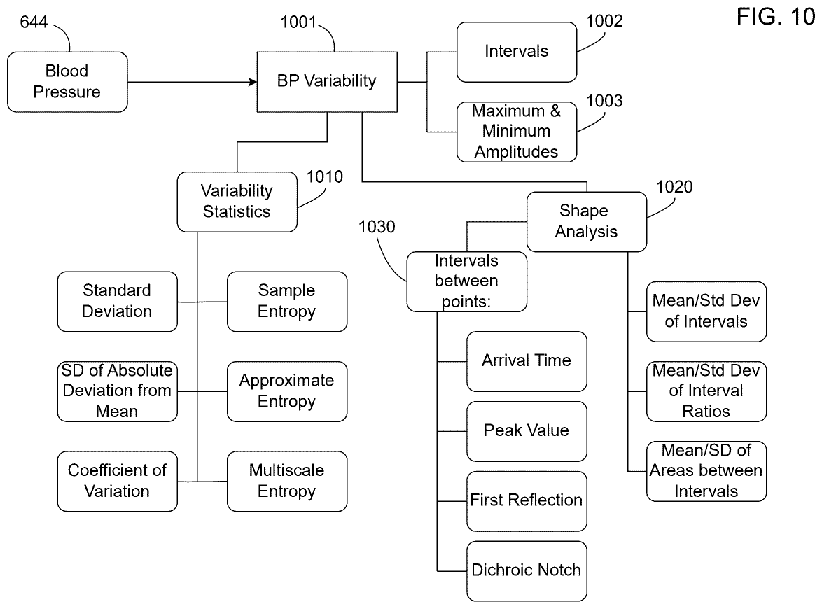

Variability measures such as those described above for heart rate variability may also be applied to any other quasi-periodic physiological functions, and input as features into the machine learning system. For example, in one or more embodiments, respiration rate variability measures may be calculated from a time series that represents breathing cycles. Blood pressure is another cyclic time series to which variability measures such as those in FIG. 9 may be applied. FIG. 10 shows additional features that may be derived from blood pressure 644, including variability features 1001 that may be applied to either or both of time intervals 1002 between blood pressure minimums and maximums, or to the minimum and maximum blood pressure values 1003 within a cycle. Variability statistics 1010 may be calculated, as for heart rate variability, over a two-minute time window, for example, and values may be standardized by dividing them by the mean value prior to calculation of variability measures. Measures that may be applied to values 1002 and 1003 may include for example standard deviations, standard deviations of absolute deviations from the mean, coefficient of variation (standard deviation divided by the mean), sample entropy, approximate entropy, and multiscale entropy. Features 1020 may characterize the shape of the blood pressure cycles, which may be based for example on time intervals 1030 between the points of arrival time, peak value, first reflection, and dichroic notch. Features derived from these intervals may include for example the mean and standard deviation of the intervals, of the ratios between intervals, and of the areas between intervals.

FIG. 11 shows illustrative features from lab results 211 and patient history and demographics 212 that may be input into a machine learning system. Lab results 211 may include any analyses of patient samples such as blood, urine, or tissue samples. Typical results that may be used include those from a basic metabolic panel and a complete blood count. Specialized lab tests may be applicable to specific machine learning systems depending on the predicted event; for example, for seizure risk a neuron specific enolase test result may be a useful feature since it is a marker of central nervous system damage. History and demographics features 212 may include typical demographics such as age, sex, race, ethnicity; basic physical features such as weight, height, and body surface area; chronic conditions such as diabetes, hypertension, substance use, and allergies; histories of any relevant injuries, diseases or conditions, such as-for seizure prediction—history of seizures, epilepsy, head trauma, and neurological infection; and any other co-morbidities. Other types of history may be relevant for prediction of different conditions or events.

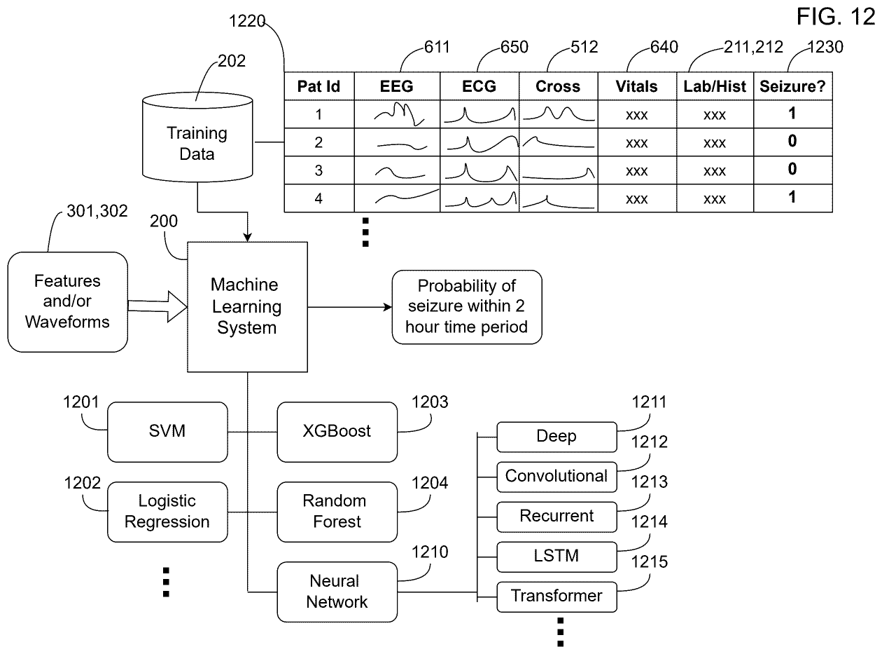

FIG. 12 shows illustrative types of machine learning technologies that may be used in one or more embodiments to implement the machine learning system 200. Illustrative technologies may include for example, without limitation, support vector machines 1201, logistic regression 1202, XGBoost 1203, random forests 1204, and various types of neural networks 1210. Neural networks may use any desired network architecture, including for example, without limitation, deep neural networks 1211, convolutional neural networks 1212, recurrent neural networks 1213, long short-term memory networks 1214, and transformer neural networks 1215.

Training data 202 for a machine learning system that predicts seizure probabilities may include for example a labeled dataset 1220 based on patient histories. The dataset may include EEG waveforms and/or extracted neurological features 611, ECG waveforms and extracted cardiovascular features 650, measures of relationships between neurological data sets and cardiovascular data sets 512, vital signs and vital sign features 640, labs and patient demographics 211 and 212, and a binary label 1230 that indicates whether the patient experienced a seizure within the prediction time period (such as 2 hours).

While the invention herein disclosed has been described by means of specific embodiments and applications thereof, numerous modifications and variations could be made thereto by those skilled in the art without departing from the scope of the invention set forth in the claims.

What is claimed is:

1. A seizure prediction system that combines brain electrical activity data and cardiorespiratory data, comprising:
one or more processors coupled to
an electroencephalogram monitor coupled to a patient and configured to measure electroencephalogram waveforms over time;
an electrocardiogram monitor coupled to said patient and configured to measure electrocardiogram waveforms over time;
one or more vital sign sensors coupled to said patient and configured to measure one or more vital signs over time;
a machine learning system configured to execute on at least one of said one or more processors and trained to calculate a probability that said patient will experience a seizure within a prediction time period;
a feature extraction system configured to execute on at least one of said one or more processors and configured to
calculate one or more features, wherein said one or more features comprise:
one or more neurological features calculated from said electroencephalogram waveforms over time;
one or more cardiovascular features calculated from said electrocardiogram waveforms over time;
one or more neurological-cardiovascular relationships calculated from one or more feature pairs, wherein each pair of said one or more feature pairs comprises:
a neurological feature of said one or more neurological features; and
a cardiovascular feature of said one or more cardiovascular features; and,
one or more vital sign features calculated from said one or more vital signs; and,
input said one or more features into said machine learning system.

2. The seizure prediction system that combines brain electrical activity data and cardiorespiratory data of claim 1, wherein said machine learning system is further trained to calculate a probability that said patient will experience within said prediction time period one or more of: sepsis, cardiogenic shock, hypotension, respiratory failure, hemorrhage, stroke, fatal arrythmia, vasospasm, and cardiac arrest.

3. The seizure prediction system that combines brain electrical activity data and cardiorespiratory data of claim 1, wherein calculation of said one or more neurological features comprises:
identify one or more discharges within said electroencephalogram waveforms; and, calculate:
a prevalence of said one or more discharges;
a duration of said one or more discharges;
a frequency of said one or more discharges;
phases of said one or more discharges;
a sharpness of said one or more discharges;
an amplitude of said one or more discharges;
a polarity of said one or more discharges; and,
an inter-discharge period between said one or more discharges.

4. The seizure prediction system that combines brain electrical activity data and cardiorespiratory data of claim 3, wherein said one or more neurological features further comprise:
a periodicity of said one or more discharges; and,
a morphology of said one or more discharges.

5. The seizure prediction system that combines brain electrical activity data and cardiorespiratory data of claim 1, wherein:
said one or more processors are further coupled to one or more cameras configured to capture video of patient movement; and,
said one or more neurological features are further calculated from said video of patient movement.

6. The seizure prediction system that combines brain electrical activity data and cardiorespiratory data of claim 1, wherein calculation of said one or more cardiovascular features comprises:
identify segments within one or more beats of said electrocardiogram waveforms over time, wherein said segments comprise a P-wave, a Q-wave, an R-wave, an S-wave, and a T-wave;
calculate amplitudes of said segments;
calculate intervals of said segments;
calculate an ST elevation from said S-wave and said T-wave;
calculate one or both of a power spectral density and principal components scores from one or both of said amplitudes of said segments and said intervals of said segments.

7. The seizure prediction system that combines brain electrical activity data and cardiorespiratory data of claim 6, wherein said one or more cardiovascular features further comprise:
an arrythmia frequency; and
one or more heart rate variability metrics.

8. The seizure prediction system that combines brain electrical activity data and cardiorespiratory data of claim 7, wherein said one or more heart rate variability metrics comprise:
a percentage of power in high frequency range;
sample entropy;
local dynamics score;
coefficient of sample entropy.

9. The seizure prediction system that combines brain electrical activity data and cardiorespiratory data of claim 1, wherein said one or more neurological-cardiovascular relationships comprise one or more of:
a correlation between said neurological feature and said cardiovascular feature;
a cross-coherence between said neurological feature and said cardiovascular feature;
a wavelet correlation between said neurological feature and said cardiovascular feature; and,
a cross-spectral entropy between said neurological feature and said cardiovascular feature.

10. The seizure prediction system that combines brain electrical activity data and cardiorespiratory data of claim 1, wherein said one or more vital signs comprise:
respiratory rate;
heart rate;
blood oxygenation;
blood pressure; and,
temperature.

11. The seizure prediction system that combines brain electrical activity data and cardiorespiratory data of claim 10, wherein said one or more vital sign features comprise:
a mean value of each vital sign of said one or more vital signs;
a median value of each vital sign of said one or more vital signs;
a standard deviation of each vital sign of said one or more vital signs;
a trend of each vital sign of said one or more vital signs;
a correlation coefficient between said heart rate and said respiratory rate; and,
a correlation coefficient between said heart rate and said blood oxygenation.

12. The seizure prediction system that combines brain electrical activity data and cardiorespiratory data of claim 10, wherein said one or more vital sign features comprise:
one or more blood pressure variability metrics, comprising a multiscale sample entropy of said blood pressure.

13. The seizure prediction system that combines brain electrical activity data and cardiorespiratory data of claim 1, wherein said one or more features further comprise:
one or more lab results associated with said patient; and,
one or more values of patient history and demographics associated with said patient.

14. The seizure prediction system that combines brain electrical activity data and cardiorespiratory data of claim 13, wherein said one or more lab results comprise:
results from a metabolic panel;
results from a complete blood count; and,
a neuron-specific enolase level.

15. The seizure prediction system that combines brain electrical activity data and cardiorespiratory data of claim 13, wherein said one or more values of patient history and demographics comprise:
age of said patient;
sex of said patient;
history of seizures of said patient;
history of epilepsy of said patient;
history of head trauma of said patient; and,
history of neurological infection of said patient.

16. The seizure prediction system that combines brain electrical activity data and cardiorespiratory data of claim 1, wherein said machine learning system comprises one or more of:
an XGBoost model;
a support vector machine model;
a logistic regression model;
a random forest model; and,
a neural network model.
